(19) United States
(12) Patent Application Publication
(10) Pub. No.: US 2017/0119936 A1
(43) Pub. Date: May 4, 2017

(54) BIODEGRADABLE WIRE WITH CENTRAL FILAMENT

(71) Applicant: FORT WAYNE METALS RESEARCH PRODUCTS CORP, Fort Wayne, IN (US)

(72) Inventors: Jeremy E. Schaffer, Leo, IN (US); Adam J. Griebel, Fort Wayne, IN (US)

(73) Assignee: FORT WAYNE METALS RESEARCH PRODUCTS CORP, Fort Wayne, IN (US)

(21) Appl. No.: 15/318,350

(22) PCT Filed: Jun. 12, 2015
(86) PCT No.: PCT/US15/35583

§ 371 Date: Dec. 12, 2016

Related U.S. Application Data
(60) Provisional application No. 62/011,703, filed on Jun. 13, 2014.
(60) Provisional application No. 62/136,023, filed on Mar. 20, 2015.

(51) Int. Cl. A61L 31/10; A61B 17/06; A61F 2/01; A61L 31/02; A61F 2/86

(52) U.S. Cl. CPC A61L 31/10; A61L 31/022; A61F 2/86; A61F 2/01; A61B 17/06166; A61F 2210/0004; A61F 2002/016; A61B 2017/00004

(57) ABSTRACT

A composite wire product includes a biodegradable parent material which forms the bulk of the cross-sectional area of the wire, and a central fiber or filament of a slower-degrading or non-biodegradable material runs throughout the length of the wire. This central filament promotes the mechanical integrity of an intraluminal appliance or other medical device made from the wire product throughout the biodegradation process by preventing non-absorbed parent material from dislodging from the central filament. Thus, the present wire design enables the creation of medical devices that are designed to improve in flexibility toward a more natural state over the course of healing, while also controlling for the possibility of non-uniform in vivo erosion.

### CROSS-REFERENCE TO RELATED APPLICATIONS

The present application claims the benefit under Title 35, U.S.C. §119(e) of U.S. Provisional Patent Application Ser. Nos. 62/011,703 and 62/136,023, filed on Jun. 13, 2014 and Mar. 20, 2015 respectively, both entitled BIODEGRADABLE WIRE WITH CENTRAL FILAMENT, the entire disclosures of which are hereby expressly incorporated by reference herein.

### BACKGROUND

1. Technical Field

The present invention relates to wires useable in medical device manufacture.

2. Description of the Related Art

Fine medical grade wire materials, such as those having the diameter of one millimeter or less, are used in a variety of medical device applications including stents, cardiac pacing leads, blood filters, and guide wires. Such wire materials may be made from corrosion resistant, non-biodegrading materials such as nickel titanium (NiTi), stainless steel, or various cobalt-chrome alloys.

Vessel wall supports, for example stents, embolic filters, and aneurysm occlusion meshes, require a degree of initial vessel wall coverage and flexural rigidity in order to hold the vessel patent and/or maintain their position. Such supports may be formed by weaving or braiding non-biodegradable wire material into a desired arrangement (e.g., a tube for a stent structure), and the support is then implanted at a desired in vivo site. In the case of stents, for example, the braided or woven tube is placed along the interior wall of an artery to alleviate arterial blockage and/or provide mechanical support to the arterial wall.

After implantation, the body drives remodeling of the local vessel architecture including resetting of the lumen to a new, usually larger size. This remodeling process causes endothelialization of the device such that once healing is complete, the device becomes integrated into the endoluminal tissue. Thus, for stents and other supports made of non-biodegradable materials, residual flexural rigidity of the device contributes to total vessel rigidity for as long as the device remains implanted.

Recent efforts have focused on providing fully absorbable in vivo devices capable of completely disappearing after therapy. Metallic solutions toward this end may utilize nutrient element metals such as Fe, Mn, Mg, Ca, Zn, and the like. For example, a biodegradable composite wire material known in the industry as “drawn filled tube” or “DFT” includes a shell material and a core material contained within the shell. In such wires, the shell and core are formed from a biodegradable wire material, such as iron, magnesium, manganese, or alloys thereof. In some cases, the core may be formed from a material which biodegrades at a faster or slower rate as compared to the shell, in order to produce a desired rate of biodegradation for the overall wire structure while also providing desired mechanical properties to the structure throughout the device life cycle in vivo.

What is needed is an improvement over the foregoing.

### SUMMARY

The present disclosure provides a composite wire product in which a biodegradable parent material forms the bulk of the cross-sectional area of the wire, and a central fiber or filament of a slower-degrading or non-biodegradable material runs throughout the length of the wire. This central filament promotes the mechanical integrity of an intraluminal appliance or other medical device made from the wire product throughout the biodegradation process by preventing non-absorbed parent material from dislodging from the central filament. Thus, the present wire design enables the creation of medical devices that are designed to improve in flexibility toward a more natural state over the course of healing, while also controlling for the possibility of non-uniform in vivo erosion.

In one form thereof, the present disclosure provides a wire material including: a filament made from a filament material; a shell surrounding the filament and having a diameter less than 1.5 mm, the shell formed from a shell material, the shell material formed from a biodegradable material having a biodegradation rate faster than the filament material, the wire defining a non-biodegraded state including both the filament and the shell and a biodegraded state including only the substantially intact filament, the wire defining a first flexural rigidity in the non-biodegraded state and a second flexural rigidity in the biodegraded state, the first flexural rigidity being at least two orders of magnitude larger than the second flexural rigidity, whereby the flexibility of the wire increases as the shell biodegrades.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a partial cross-section, perspective view of a wire made in accordance with the present disclosure in an as-manufactured form;

FIG. 2 is a partial cross-section, perspective view of the wire of FIG. 2, after early-stage in vivo biodegradation;

FIG. 3 is a partial cross-section, perspective view of the wire of FIG. 2, after advanced in vivo biodegradation;

FIG. 5a is a cross-section, perspective view of a prior art monolithic biodegradable wire;

FIG. 5b is a cross-section, perspective view of a wire made in accordance with the present disclosure, including a central filament;

FIG. 5c is a cross-section, perspective view of another wire made in accordance with the present disclosure, including a central filament with a reduced diameter;

FIG. 6a is a cross-section, perspective view of another wire made in accordance with the present disclosure, including multiple central filaments arranged in parallel;

FIG. 6b is a cross-section, perspective view of another wire made in accordance with the present disclosure, including multiple central filaments arranged into a multi-strand twisted cable;

FIG. 7 is a schematic view illustrating an exemplary forming process of wire using a lubricated drawing die;

FIG. 8 is a perspective view of a braided stent made with wire of the present disclosure; and

FIG. 9 is a perspective view of a woven stent made with wire of the present disclosure.

Corresponding reference characters indicate corresponding parts throughout the several views. Although the exemplifications set out herein illustrate embodiments of the invention, the embodiments disclosed below are not intended to be exhaustive or to be construed as limiting the scope of the invention to the precise form disclosed.

### DETAILED DESCRIPTION

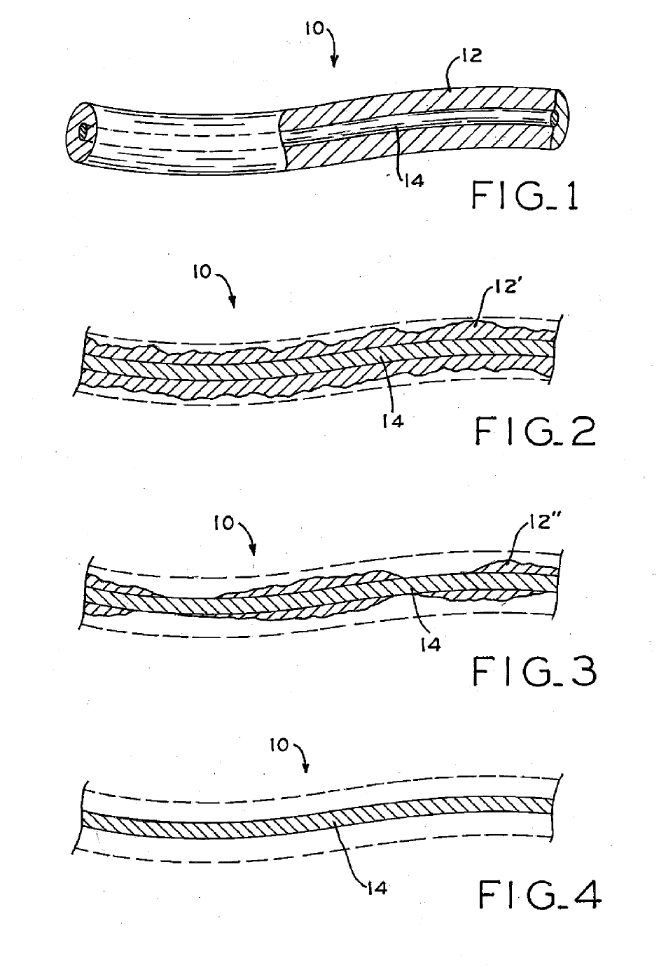

The present disclosure provides wire 10, shown in FIG. 1, including shell 12 made from a biodegradable metal or metal alloy and central filament 14 disposed within the shell and made from a non-biodegrading, or slowly biodegrading material. As the material of shell 12 degrades after implantation in a patient's body, central filament 14 acts as a scaffold or support which binds to shell 12 along the axial extent of wire 10, ensuring that degraded portions of shell 12 are held in place by adherence (e.g., by chemical bonding and/or mechanical fixation) to filament 14, even if such degraded portions are otherwise unconnected to the rest of wire 10. This retention of shell 12 upon filament 14 inhibits formation of embolic debris while maintaining the major benefits of absorbable technology, including an initially stiff construct which provides flexural rigidity to enable acute therapy by providing initial vessel support, similar to existing permanent vascular stent platforms. As the shell is absorbed, and in the event of non-uniform surface erosion, the central filament or plurality of filaments engages with and entrains the debris until they are eliminated or substantially reduced in size.

As described in detail below, the material and cross-sectional size of central filament 14 are chosen such that only a thin, flexible framework remains in the body after shell 12 has fully degraded. This framework has a minimal, near-zero impact on total vessel rigidity, and may therefore be considered to be substantially “mechanically transparent” to the surrounding tissue in that the mechanical effect of the filament framework is negligible in the context of the mechanical characteristics of the vessel itself. In addition, the material of the framework may be chosen such that the framework itself slowly degrades to an eventual zero impact. In an exemplary embodiment described further below, wire 10 (and by extension, any device made using wire 10) defines an as-manufactured, non-biodegraded state when shell 12 is received over filament 14 and fully intact. In a biodegraded state, shell 12 is completely absorbed and only filament 14 remains. The size and material of filament 14 is chosen such that the flexibility of the device and thus the treated vessel anatomy is improved by at least two orders-of-magnitude. This process and the mathematical relationships are further described below.

For purposes of the present disclosure, the terms “bioabsorbable,” “bioresorbable” and “biodegradable” are used interchangeably to indicate materials which are able to be chemically broken down in a physiological environment, i.e., within the body or inside body tissue, by processes such as resorption or absorption, over a known and/or controlled period of time. For example, medical appliances made of biodegradable materials in accordance with the present disclosure will generally completely degrade within a period of weeks to months, such as 18 months or less, 24 months or less, or 36 months or less, for example. This rate stands in contrast to more “degradation-resistant” or permanent appliances, such as those constructed from NiTi or stainless steel, which remain in the body, structurally intact, for a period exceeding at least 36 months and potentially throughout the lifespan of the recipient. Biodegradable metals used herein include nutrient metals, i.e., metals such as iron, magnesium and manganese, all of which have biological utility and are used by, or taken up in, biological pathways.

Conversely, “non-bioabsorbable,” “non-bioresorbable” or “non-biodegradable” materials are those which are not able to corrode appreciably in vivo over the lifetime of a person. For example, materials which can be expected to lose less than 1% of their mass to corrosion over the course of a human lifetime can be considered non-biodegradable for purposes of the present disclosure. Non-biodegradable materials may also be defined in terms of their ion release rates in an in vivo environment. For example, non-biodegradable materials define in vivo ion release rates on the order of tens of parts per million or less up to several hundred parts per million per year, but not exceeding about 1,000 parts per million per year. Generally speaking, “non-biodegradable” material in the present disclosure is material that biodegrades at a rate commensurate with the materials above. Yet another definition of a non-biodegradable material in accordance with the present disclosure is a material which can be expected to remain at least partially intact for a period of at least ten years in vivo, as could be verified by, e.g., post-mortem examination, x-ray imaging, magnetic resonance imaging (MRI), and other common imaging and inspection techniques.

“Slowly bioabsorbable,” “slowly bioresorbable,” or “slowly biodegradable” materials are those which can be expected to degrade more quickly than non-biodegradable materials in vivo, but also more slowly than biodegradable materials in vivo. As further described below, slowly biodegradable materials may be used for potions of a wire or medical device which remains substantially intact in vivo after the period of weeks to months during which the biodegradable materials are absorbed or resorbed, but are then slowly absorbed or resorbed over a period of months to years.

As used herein, “elastic modulus” is defined as Young's modulus of elasticity and is calculated from the linear portion of the tensile, monotonic, stress-strain load curve using linear extrapolation via least squares regression, in accordance with ASTM E111. Units are stress, in gigapascals (GPa).

“OD” refers to the outside diameter of a metallic wire or outer shell.

“ID” refers to the inside diameter of a metallic outer shell.

1. Biodegradable, Bioresorbable and Bioabsorbable Materials

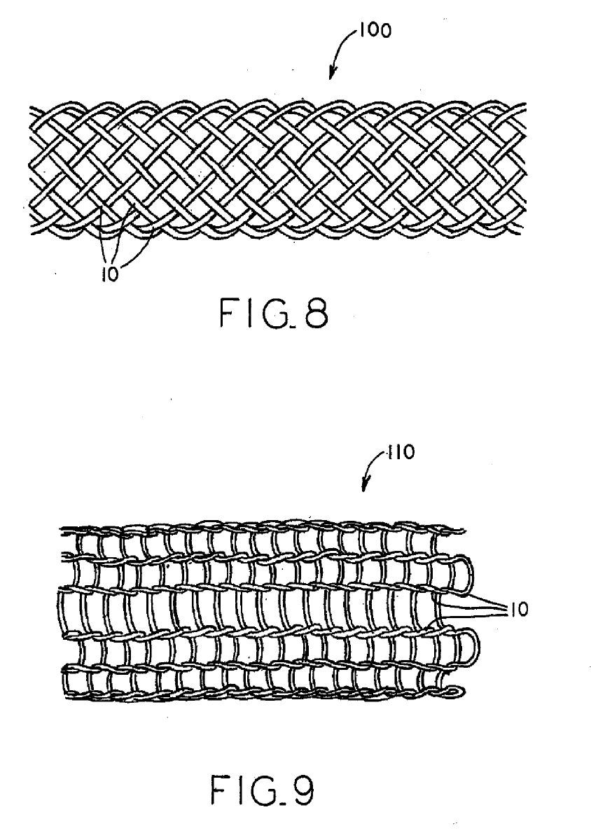

Biodegradable, nutrient metal alloys can be used to form fine medical grade wires 10 into medical devices such as braided and woven stents 100, 110 as shown in FIGS. 8 and 9 respectively. After implantation, shell 12 of wire 10 (FIG. 1), which forms a majority of the material of wire 10 as described further below, slowly biodegrades within the body. In particular, the nutrient metal materials of shell 12 are carried away by the bloodstream and incorporated into the body of the patient by resorption or absorption.

The constituent elements of a biodegradable wire material made in accordance with the present disclosure may be produced by material processing techniques which create a wire which will biodegrade at a specified rate, such that the resulting medical device will remain present for a specified period of time at the implanted location, and will have specified mechanical characteristics (e.g., strength, ductility, etc.) over the device life cycle. Thus, rather than utilizing a second surgical procedure to remove the device or allowing the device to remain implanted, the device material is allowed to slowly biodegrade. After a period of time, the device may be substantially or entirely eliminated, such that the vessel is permitted to resume normal, unaided function.

Bioabsorbable materials and alloys suitable for use with the present wire constructs are described in U.S. Patent Application Publication No. 2011/0319978 filed Jun. 24, 2011 and entitled BIODEGRADABLE COMPOSITE WIRE FOR MEDICAL DEVICES, International Patent Application Serial No. PCT/US2014/041267 filed Jun. 6, 2014 and entitled BIODEGRADABLE WIRE FOR MEDICAL DEVICES, and International Patent Application Serial No. PCT/US2013/049970 filed Jul. 10, 2013 and entitled BIODEGRADABLE ALLOY WIRE FOR MEDICAL DEVICES, all of which are commonly owned with the present application, the entire disclosures of which are hereby expressly incorporated herein by reference.

2. In Vivo Biodegradation of Composite Wire Constructs

Turning now to FIG. 1, wire 10 in accordance with the present disclosure is shown in a non-biodegraded state, after initial manufacture. This illustrated configuration of wire 10 is the same configuration present just after implantation within a human body (i.e., total implanted time t=0). As illustrated, wire 10 includes a fully intact, substantially cylindrical shell 12 in its as-manufactured state, which has an uninterrupted, smooth outer surface and a round cross-section. In other embodiments, non-round cross sectional shapes may be used including polygons, ovals and the like as may be required or desired for a particular application. To the extent that the outer surface of shell 12 defines the same cross-sectional shape at all points along its axial length, shell 12 can be considered to be non-biodegraded, “whole” and “uninterrupted.”

At time t=0, the structure into which wire 10 is incorporated (e.g., one of stents 100, 110, or another medical device as described below) has a flexural rigidity R defined as the product of Young's elastic modulus, E, for wire 10 and the second moment of area, I, for the chosen cross-section of wire 10. That is, R=E*I. For time t=0, the flexural rigidity R may be labeled R₀, and the second moment of area may be labeled I₀, reflecting that the entire shell 12 and filament 14 are intact (i.e., non-biodegraded) and behave together as a bonded whole.

For wire 10 having a round cross-section comprised of a centrally located round filament 14 coaxial with its surrounding shell 12, as shown in FIG. 1, wire 10 can be considered a circular beam which bends about a transverse axis, the typical form of deformation in wire flexure. On these assumptions, the second moment of area I for wire 10 is:

$$I = π ( \frac{{d}^{4}}{64} ) , \qquad ( Eq . 1 )$$

with I₀ corresponding to the finished diameter D_{2S} of wire 10 at time t=0.

Therefore, flexural rigidity R can be expressed as follows:

$$R = EI = π ( \frac{{Ed}^{4}}{64} ) \qquad ( Eq . 2 )$$

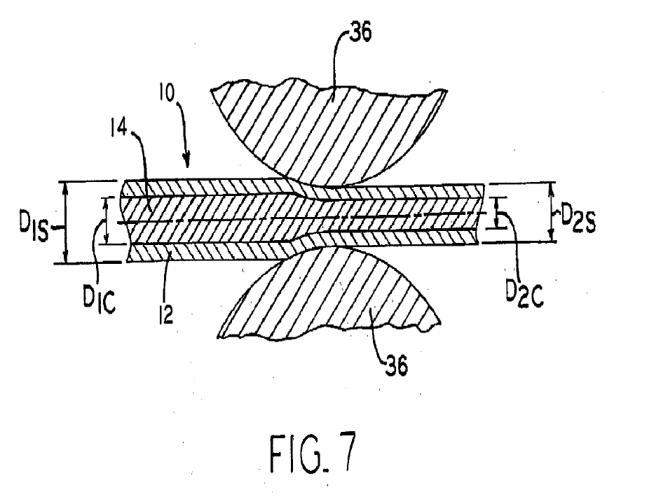

For wire 10, flexural rigidity R is also impacted by the elastic modulus E, which in turn is derived from a composite of the elastic moduli of both shell 12 and filament 14. If shell 12 has an elastic modulus E_{S} and diameter d_{S} (equivalent to finished diameter D_{2S} shown in FIG. 7), and filament 14 has an elastic modulus E_{F} and diameter d_{F} (equivalent to finished diameter D_{2C} shown in FIG. 7) the resulting flexural rigidity R₁ can be expressed as:

$${R}_{1} = \frac{π}{64} ( {E}_{S} ( {d}_{S}^{4} - {d}_{F}^{4} ) + {E}_{F} {d}_{F}^{4} ) \qquad ( Eq . 3 )$$

Turning to FIG. 2, wire 10 is shown after having been implanted for a period of time t=1. At the illustrated time t=1, the material of shell 12 has begun to biodegrade, i.e., by resorption or absorption of molecules from the outer surface of shell 12 into the bloodstream of the patient. This partially-degraded shell is depicted as shell 12′. As noted above, initial biodegradation of shell 12′ may result in erosion of the outer surface of shell 12′ in a non-uniform manner, as illustrated schematically in FIG. 2. However, although some of the material of shell 12′ has disappeared from the outer surface of the original structure of wire 10, the biodegradable shell 12′ still completely surrounds the non-biodegradable central filament 14.

FIG. 3 shows wire 10 at time t=2, which is later than time t=1. As illustrated, further degradation has occurred as the material of shell 12′ is resorbed or absorbed into the bloodstream. This further degraded shell 12′ is depicted as shell 12″. Some sections of shell 12″ are shown to be eroded down to filament 14, such that the non-biodegradable or slowly biodegrading material of central filament 14 is directly exposed to the bloodstream. Remaining sections of non-biodegraded material of shell 12″ remain bonded to the central filament. In particular, the initial drawing of wire 10 creates a microstructurally complex surface interaction between shell 12 and filament 14, due to roughness of the material surfaces, slight irregularities among the surfaces, and the like. This creates a micro-interlocking, friction-type fit between shell 12 and filament 14 which persists as shell 12 degrades to shell 12′ and shell 12″. In some embodiments, diffusion bonding or other metallurgical bonding may also contribute to the adherence of shells 12, 12′, 12″ to filament 14. Advantageously, the presence of the non-biodegradable central filament 14 and its bond to shell 12, 12′ and/or 12″ reduces the possibility of the otherwise isolated sections of biodegradable material dislodging from the overall structure, thereby reducing the possibility any such dislodged material traveling within the bloodstream prior to completion of the biodegradation process.

At a still later time t=3 shown in FIG. 4, all of the biodegradable material has been resorbed or absorbed into the patient's bloodstream, leaving only the non-biodegradable or slowly degrading filament 14 remaining in the original medical device structure. At this point, the mechanical and biodegradation properties of the medical device into which wire 10 was originally incorporated are controlled entirely by filament 14. Thus, the flexural rigidity R₂ of wire 10 and, therefore, of the entire medical device made from wire 10, will be lower than the original flexural rigidity R₁ described in detail above. For purposes of the following discussion, it will be assumed that shell 12 is completely biodegraded and filament 14 remains fully intact, though it is appreciated that filament 14 may also degrade at a relatively slower pace as compared to shell 12 as further discussed below.

Thus, for wire 10 with no remaining shell 12 and an intact filament 14, flexural rigidity R₂ can be expressed as:

$${R}_{2} = \frac{π}{64} ( {E}_{F} {d}_{F}^{4} ) \qquad ( Eq . 4 )$$

The difference between the initial flexural rigidity R₁ at time t=0 and the final flexural rigidity R₂ at time t=3 may be controlled by design of wire 10, including choice of material and geometric wire design as further described below. For purposes of the present disclosure, comparison factor F may be defined to describe the comparative reduction of rigidity R from rigidity R₁ at time t=0 and rigidity R₂ at time t=3. For example, rigidity comparison factor F may be expressed as R₁/R₂, derived from dividing Eq. 3 by Eq. 4. Thus, it may be stated that the “compliance” or “flexibility” of wire 10 (and of a medical device made from wire 10) is “improved” by a factor of R1/R2 as time t=3 and the associated complete degradation of shell 12 is achieved. In particular, the factor F of improvement in flexibility may be expressed as:

F=R₁/R₂=(Eₛ(dₛ⁴−d_{f}⁴)+E_{f}d_{f}⁴)/(E_{f}d_{f}⁴) (Eq. 5)

In one embodiment, wire 10 may include shell 12 and filament 14 respectively made from materials having equal or near-equal moduli of elasticity. In this case, factor F or the “improvement in flexibility” may be expressed as a function of wire geometry alone and independent of the moduli of particular materials used. This geometry-based factor is expressed as F_{GEOMETRIC} as follows:

F=_{geometric}=(dₛ/d_{f})⁴ (Eq. 6)

In another embodiment, wire 10 may include shell 12 with a relatively small-diameter (or cross-sectional area) for filament 14, such that the contribution of filament 14 to the initial rigidity of wire 10 is negligible. In this case, the moduli of elasticity may be the only significant factor in determining factor F. This material-based factor is expressed as F_{MATERIAL} as follows:

Fₘₐₜₑᵣᵢₐₗ=E_{S}/E_{f} (Eq. 7)

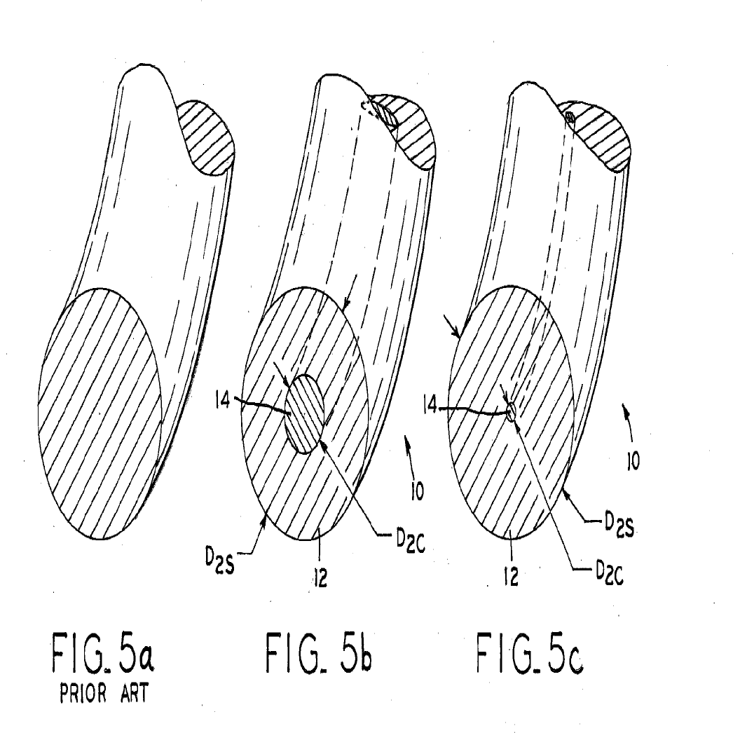

In the illustrated embodiments of FIGS. 1, 5b and 5c, wire 10 includes a coaxial shell 12 and filament 14. That is, shell 12 and filament are concentric, such that thickness T of shell 12 is constant around the entire periphery of filament 14. In such a wire construct, core ratio X may be defined as the ratio of the cross-sectional area of central filament 14 to the sum of the areas of both shell 12 and filament 14 (i.e., the total initial area of wire 10). Core ratio X is as follows:

X=(d_{f}/dₛ)² (Eq. 8)

As described in further detail below, in certain exemplary embodiments, shell 12 of wire 10 will be provided with a modulus generally higher than the modulus of filament 14, such that shell 12 provides initial a high initial rigidity (such as to maintain or restore vessel patency in a stent application) while filament 14, after shell biodegradation and dissolution, provides lesser rigidity at a later time. Moreover, core ratio X, the materials chosen for shell 12 and filament 14, and the overall size and geometry of wire 10 may be controlled to provide for a particular factor F of improvement in flexibility between time t=0 and time t=3, with factor F gradually (if not necessarily linearly) increasing after initial implantation at time t=0 until full dissolution of shell 12 at time t=3.

In an exemplary embodiment, the flexural rigidity of wire 10 in its non-biodegraded state (i.e., at time t=0) is orders of magnitude higher than the flexural rigidity of wire 10 in it biodegraded state (i.e., at time t=3). For example, factor F may be at least two, three or four orders of magnitude, or may be as much as five, six or seven orders of magnitude, or may be any differential between the flexural rigidities at times t=0 and t=3 within any range defined by any pair of the foregoing values, such as, for example, two to seven orders of magnitude, three to six orders of magnitude, or four to five orders of magnitude. Although the specific materials chosen for shell 12 and filament 14 have a significant effect on factor F, an exemplary embodiment will utilize a finished diameter D_{2C} of filament 14 of as little as 5%, 10% or 15% of shell diameter D_{2S}, or as large as 35%, 45% or 50% of shell diameter D_{2S} or any percentage within any range defined by any pair of the foregoing values, such as, for example, 5% to 50%, 10% to 45% or 15% to 35%. Generally speaking, setting filament diameter D_{2C} at less than one-half of shell diameter D_{2S} can cooperate with material choices to ensure the desired differential of rigidity between the non-biodegraded and biodegraded states.

Optionally, shell 12 may include intentional interruptions formed in its outer surface, such as etchings or machined imperfections, to serve as a nucleation site for corrosion of shell 12 in vivo. For example, a “hinge” or other intentionally flexible portion can be designed in to the medical device by inducing corrosion at the intentional interruption, such that flexural rigidity falls much lower at one portion of the device at times t=1 and/or t=2 as compared to the other, non-interrupted portions of the device. Wire 10 in accordance with the present disclosure is uniquely suited to this type of application, because filament 14 can be designed to remain intact as long as necessary (e.g., by using a very slowly biodegrading, or non-biodegrading material) to ensure endothelialization, even other portions of shell 12 have not yet not degraded.

Conversely, portions of wire 10 may be protected from early degradation in order to promote or induce a particular degradation profile along the axial extent of wire 10. For example, an anti-degradation coating, such as oxides, polymers or ceramics, may be applied to portions of the outer surface of shell 12 upon manufacture of wire 10, such that the coated portions will experience slower degradation in vivo as compared to uncoated portions. In one exemplary embodiment, polymer coatings may include biodegradable polymers such as polyglycolic acid (PGA), polylactic acid (e.g., PLLA), or a copolymer thereof.

In another optional embodiment of the present wire 10, filament 14 may be formed from nickel-titanium material capable of being thermally shape set. This shape setting process may be performed on filament 14 to place filament in a first configuration, at which point filament 14 is integrated into wire 10 while in one of its two thermally-variable states. Shell 12 may be provided with sufficient strength and rigidity to be unaffected by changes in the natural geometry of filament 14 arising from changes in the ambient temperature, such that the geometry of shell 12 effectively controls the overall geometry of wire 10. Shell 12, and therefore wire 10, may be placed in a second configuration different from the first configuration such that filament is elastically deformed into the first configuration, but as shell 12 degrades, the NiTi filament 14 will be allowed to regain its shape-set first configuration.

3. Composite Wire Constructs Including a Shell and Central Filament

To form wire 10, filament 14 is inserted within shell 12 to form a pre-drawn wire construct, and an end of the wire construct is then tapered to facilitate placement of the end into a drawing die. The end protruding through the drawing die is then gripped and pulled through the die to reduce the overall diameter of the construct, which also brings filament 14 into firm physical contact with shell 12 along their respective axial extents. After drawing, the inner diameter of shell 12 closes on the outer diameter of filament 14 such that the inner diameter of shell 12 equals the outer diameter of filament 14 whereby, when viewed in section, the inner filament will occupy and completely fill the central void of outer shell 12.

The step of drawing wire 10 subjects the material to cold work. More particularly, drawing imparts cold work to the material of both shell 12 and filament 14, with concomitant reduction in the cross-sectional area of both materials. The total cold work imparted to the material during the drawing step can be characterized by the following formula (I):

$$cw = 1 - {( \frac{{D}_{2}}{{D}_{1}} )}^{2} \qquad ( I )$$

wherein “cw” is cold work defined by reduction of the original material area, “D₂” is the diameter of the wire after the draw or draws, and “D₁” is the diameter of the wire prior to the same draw or draws. In FIG. 7, “D₁” is shown as “D_{1S}” for shell 12 and “D_{1C}” for filament 14 and, similarly, “D₂” is shown as “D_{2S}” for shell 12 and “D_{2C}” for filament 14.

Referring to FIG. 7, the cold work step is performed by drawing wire 10 through a lubricated die 36 having an output diameter D_{2S}, which is less than diameter D_{1S} of the undrawn wire 10. Although drawing is one exemplary method of imparting cold work to wire 10, other methods may be used as required or desired for a particular application. For example, wire 10 may be cold-swaged, rolled flat or into other shapes which result in the net accumulation of cold work. Cold work may also be imparted by any combination of techniques including the techniques described here, for example, cold-swaging followed by drawing through a lubricated die finished by cold rolling into a ribbon or sheet form or other shaped wire forms.

In one embodiment, the cold work step by which the diameter of wire 10 is reduced from D_{1S} to D_{2S} is performed in a single draw and, in another embodiment, the cold work step by which the diameter of wire 10 is reduced from D_{1S} to D_{2S} is performed in multiple draws which are performed sequentially without any annealing step therebetween. In multiple-draw processing, the drawing process is repeated, with each subsequent drawing step further reducing the cross section of wire 10 proportionately, such that the ratio of the sectional area of filament 14 to the overall sectional area of wire 10 is nominally preserved as the overall sectional area of wire 10 is reduced. Referring to FIG. 7, the ratio of pre-drawing core outer diameter D_{1C} to pre-drawings shell outer diameter D_{1S} is the same as the corresponding ratio post-drawing. Stated another way, D_{1C}/D_{1S}=D_{2C}/D_{2S}. In wires 10 where filament 14 is a polymer and shell 12 is a metal material, filament 14 may experience a small amount of initial compression but quickly becomes effectively incompressible, such that the conservation of relative volumes of filament 14 and shell 12 remains in accordance with the above equation.

Thermal stress relieving, otherwise known in the art as annealing, at a nominal temperature not exceeding the melting point of either the first or second materials, may be used to improve the ductility of the fully dense composite between drawing steps, thereby allowing further plastic deformation by subsequent drawing steps.

The softening point of the present materials is controlled by introducing cold work into the composite structure after joining the metals. Deformation energy is stored in the structure which serves to reduce the amount of thermal energy required for stress relief. For wires 10 having shell 12 made of iron or iron alloys, cold work processing facilitates annealing of the composite structure at temperatures in the range of 40 to 50% of the melting point of shell 12, in a manner sufficient to provide ductility to both metal species and successful fine wire production. Such ductility also facilitates spooling of the wire, as discussed below, and renders the wire suitable for in vivo uses where low ductility would be undesirable.

Additional details regarding the manufacture of composite wires with non-biodegradable shells and cores can be found in various references describing “drawn filled tubing” or DFT structures, including U.S. Pat. Nos. 7,420,124, 7,501,579 and 7,745,732, the entire disclosures of which are hereby expressly incorporated herein by reference for all that they teach and for all purposes.

Exemplary non-biodegradable materials for central filament 14 include stainless steel, tantalum, nickel titanium (also known as NiTi or Nitinol), Co—Ni—Cr—Mo alloy (also known as 35 NLT, or ASTM F562 material), platinum, palladium, titanium, beta-titanium (for example, Ti Beta C which is nominally 3% aluminum, 8% vanadium, 6% chromium, 4% molybdenum, 4% zirconium and balance titanium), and alloys thereof. In some cases, filament 14 may be a high strength non-biodegradable polymer.

As noted above, central filament 14 may also be made from a material which biodegrades relatively slowly, as compared to shell 12 made of a faster-degrading material. Exemplary materials with relatively low rates of degradation in vivo include iron and zinc. Fe—Mn alloys have suitably low rates of degradation for use as central filament 14 in wires 10 including shell 12 made from Mg, which has a relatively higher rate of degradation. In another exemplary combination, Mg or Mg alloy may be used for shell 12 where a slower-degrading Mg alloy is used for filament 14. Yet further exemplary combinations include a wire 10 with shell 12 made from Mg and filament 14 made from Zn, a wire 10 with shell 12 made from Fe and filament 14 made from W (it being understood that tungsten is very slowly absorbable in vivo).

In an exemplary embodiment, the rates of biodegradation for shell 12 and filament 14 are set such that shell 12 will completely disappear before filament 14 experiences any significant degradation. This ensures that filament 14, or a matrix of filaments 14 as may be provided in some medical devices, will be reliably intact throughout most or all of the degradation process of shell 12. For example, the material and geometry of shell 12 may be chosen such that shell 12 substantially completely biodegrades before filament 14 loses more than 5% of its mass, such that filament 14 can be expected to reliably retain shell 12 from dislodging from filament 14 during the entire degradation process of shell 12. In order to promote this substantially complete biodegradation of shell 12 prior to any significant loss of mass in filament 14, the total expected time for in vivo biodegradation of shell 12 may be a fraction of the total expected time for in vivo biodegradation of filament 14. For example, the expected in vivo degradation time of shell 12, expressed as a percentage of the expected in vivo degradation time of filament 14, may be as little as 2%, 10% or 15%, or as much as 20%, 25% or 30%, or may be any percentage within any range defined by any pair of the foregoing values, such as, for example, 2% to 30%, 10% to 25% or 15% to 20%.

Exemplary materials for shell 12 include ZM21 (a medium-strength forged Magnesium alloy nominally comprising 2 wt % Zn, 1 wt % Mn and a balance of Mg), WE43 (magnesium alloys nominally comprising 4 wt. % yttrium, 3 wt. % rare earths, 0.5 wt. % zirconium, balance magnesium, as set forth in ASTM B107-13), Mg and its alloys, Fe, Fe—Mn and Zn. Additional biodegradable materials suitable for shell 12 are disclosed in U.S. Patent Application Publication No. 2011/0319978 filed Jun. 24, 2011 and entitled BIODEGRADABLE COMPOSITE WIRE FOR MEDICAL DEVICES, International Patent Application Serial No. PCT/US2014/041267 filed Jun. 6, 2014 and entitled BIODEGRADABLE WIRE FOR MEDICAL DEVICES, and International Patent Application Serial No. PCT/US2013/049970 filed Jul. 10, 2013 and entitled BIODEGRADABLE ALLOY WIRE FOR MEDICAL DEVICES, all of which are commonly owned with the present application, the entire disclosures of which are hereby expressly incorporated herein by reference for all that they teach and for all purposes.

Particular exemplary embodiments in accordance with the present disclosure are shown in FIGS. 5b and 5c. In FIG. 5a, for comparison, a prior art monolithic wire having an outer diameter of 200 μm is illustrated. In FIG. 5b, wire 10 made in accordance with the present disclosure is shown, with central filament 14 having diameter D_{2C} surrounded by shell 12 having thickness T and diameter D_{2S}. In an exemplary embodiment, filament 14 is made of tantalum (Ta) having a diameter of 64 μm, and is centrally located within the 68-μm thick Fe—Mn shell (i.e., shell 12 and filament 14 are coaxial), such that the overall wire construct of FIG. 5b has a diameter D_{2S} of 200 μm. In an alternative exemplary embodiment, filament 14 is made of Nitinol (NiTi) having a diameter of 64 μm, and is centrally located within the 68-μm thick Fe—Mn shell (i.e., shell 12 and filament 14 are coaxial), such that the overall wire construct of FIG. 5b has a diameter D_{2S} of 200 μm.

In FIG. 5c, a further wire 10 made in accordance with the present disclosure is shown, which is similar to the wire construct of FIG. 5b in overall size and geometry but has filament 14 having a smaller diameter D_{2C}′ as compared to diameter D_{2C} of FIG. 5b. In exemplary embodiments, the area of wire 10 occupied by filament 14, expressed as a percentage of the overall area of wire 10, may be as little as 1%, 3%, 4% or 5%, or may be as much 6%, 10%, 15% or 20%, or filament 14 may occupy any percentage of the area of wire 10 within any range defined by any of the foregoing values, such as, for example, 1% to 20%, 3% to 15%, 4% to 10%, or 5% to 6%. Similarly, and within a given set of constraints on the overall design of wire 10 in view of moduli EF, ES, factor F, and other variables as described herein, the overall diameter of wire 10, expressed as diameter D_{2S} of shell 12, may be as small as 15 μm, 35 μm, 50 μm or 75 μm, or as large as 100 μm, 300 μm, 500 μm or 1.5 mm, or may be any diameter within any range defined by any of the foregoing values, such as, for example, 15 μm to 1.5 mm, 35 μm to 500 μm, 50 μm, to 300 μm, or 75 μm to 100 μm.

In certain exemplary embodiments, such that for use of wire 10 in in vivo medical devices as described below, the modulus of elasticity of the material of shell 12 may range from as little as 40 GPa (e.g. magnesium), 60 GPa or 80 GPa to as much as 190 GPa, 210 GPa, or 230 GPa (e.g. iron, steel, Fe—Mn), or may have any modulus within any range defined by any of the foregoing values, such as, for example, 40 GPa to 230 GPa, 60 GPa to 210 GPa or 80 GPa to 190 GPa. The modulus of elasticity of the material of filament 14 may range from as little as 0.5 GPa (e.g. polymer), 20 GPa or 40 GPa to as much as 190 GPa, 210 GPa, or 230 GPa (e.g. CoNiCrMo, iron, steel, tantalum), or may have any modulus within any range defined by any of the foregoing values, such as, for example, 0.5 GPa to 230 GPa, 20 GPa to 210 GPa, or 40 GPa to 190 GPa.

Table 1 provides a number of design parameters for achieve desired factors F of improvement in flexibility. In exemplary embodiments, and depending on the intended use of wire 10, the factor F of improvement in flexibility is designed to range from just over one order-of-magnitude (e.g., F=19 in Config. 6 of Table 1), to greater than six-orders-of-magnitude (e.g., F=4.6 million in Config. 1 of Table 1). In an exemplary embodiment of wire 10, such as in connection with its use in a medical device as described herein, factor F is at least two orders-of-magnitude, such that wire 10 provides a substantially lower flexural rigidity and a substantially “mechanically invisible” structure at its in vivo implantation site after shell 12 has biodegraded but filament 14 remains substantially intact.

Although the cases listed in Table 1 and their associated ranges of factor F and other variables does not represent an exhaustive list of materials and constructions in accordance with the present disclosure, Table 1 defines a range of design parameters for wire 10 which achieve a combination of flexibility enhancement through degradation of shell 12, while also securely retaining the material of shell 12 throughout the degradation process.

TABLE 1
Boundary cases for shell and filament modulus and geometry

| | | | core | | | | | | |
|---|---|---|---|---|---|---|---|---|---|
| | | | ratio | | ds | df | F | F | |
| Config. | Ef | Es | (X) | ds/df | (mm) | (mm) | (Eq. 6 × Eq. 7) | (Eq. 5) | error |
| 1 | 0.5 | 230 | 1% | 10.00 | 100 | 10.0 | 4600000 | 4599541 | 0.0% |
| 2 | 0.5 | 230 | 5% | 4.47 | 100 | 22.4 | 184000 | 183541 | 0.3% |
| 3 | 0.5 | 230 | 10% | 3.16 | 100 | 31.6 | 46000 | 45541 | 1.0% |
| 4 | 220 | 40 | 1% | 10.00 | 100 | 10.0 | 1818 | 1819 | 0.0% |
| 5 | 220 | 40 | 5% | 4.47 | 100 | 22.4 | 73 | 74 | −1.1% |
| 6 | 220 | 40 | 10% | 3.16 | 100 | 31.6 | 18.2 | 19.0 | −4.3% |

A further inference may be drawn from Table 1 concerning the factor F of improvement in flexibility. More particularly, Table 1 illustrates the “error rate” or difference between calculations of factor F by two methods. The first method is simply using Eq. 5 while the second method is a multiplication of Eq. 6 and Eq. 7. The very low error rate shown in Table 1 demonstrates that factor F may be estimated simply by Eq. 9 below, which is a multiplication of Eq.'s 6 and 7, as follows:

F≈Fₘₐₜₑᵣᵢₐₗ×F_{geometric}=(Eₛ/E_{f})(dₛ/d_{f})⁴ (Eq. 9)

Over the range of anticipated designs for wire embodied by the parameters set forth in Table 1, it can be seen that Eq. 9 provides a good estimate of F with less than 5% error. This equation also quantifies the dominance of geometric factor F_{GEOMETRIC} because of the quartic versus linear dependence.

A filament 14 selected with a twice-as-flexible material factor F as compared to shell 12 (i.e., filament 14 has a lower young's modulus of elasticity as compared to shell 12), gives a two-fold improvement in the material-based factor F (see also, Eq. 7 above):

Fₘₐₜₑᵣᵢₐₗ=Eₛ/E_{f}=200/100=2 (Eq. 10)

One exemplary embodiment of wire 10 having filament 14 that is twice as flexible as shell 12 can be created, for example, with an Fe—Mn shell (E≈200 GPa) and a beta titanium filament (E≈100 GPa). Where diameter D_{2C} of filament 14 is set at one half of the overall shell diameter D_{2S} (i.e., wire 10 has a core ratio of 25%), a 16-fold improvement by geometry is achieved as shown in the following:

F_{geometric}=(dₛ/d_{f})⁴=(100/50)⁴=16 (Eq. 11)

Thus, the total flexibility improvement factor F can be estimated by Eq. 9 for wire 10 having an Fe—Mn shell and a beta titanium core with a core factor of 25%. In particular, F_{MATERIAL}×F_{GEOMETRIC}=2×16=32. Thus, once shell 12 is fully biodegraded in this construction of wire 10, wire 10 can be expected to be 32 times more flexible (i.e., wire 10 will have a flexural rigidity reduced by a factor of 32) as compared to initial implantation of wire 10 with shell 12 fully intact.

Desired factors F for wire 10 may be controlled based on the intended end use of wire 10. For example, in the case of a stent (such as stents 100, 110 shown in FIGS. 8 and 9), initial flexural rigidity R₀ is dictated by the desired level of vessel wall support needed upon initial implantation, while final flexural rigidity R₂ may be minimized within the bounds of providing adequate mechanical support to shell 12 throughout degradation. Thus, the respective wires 10 used in the stent may degrade to include only filaments 14 over a specified time, at which point filaments 14 may be allowed to undergo endothelialization. Because filaments 14 have a low flexural rigidity, their impact on the mechanics and overall function of the vessel wall is minimized. Exemplary stent embodiments are discussed in further detail below.

In one embodiment of the present disclosure, the material and mechanical properties of the filament may be chosen so that the final structure of the medical device approximates the mechanical properties of the adjacent tissue and can therefore be described as “mechanically invisible” to the body. For example, in the case of a stent, the woven stent structure may provide high initial strength when the biodegradable material is present in its non-biodegraded, as-manufactured form (e.g., at time t=0), and may undergo a steady reduction in strength, flexural rigidity and other the mechanical properties during the biodegradation process. When biodegradation of shell 12 is complete (e.g., at time t=3), the remaining woven stent structure including only the substantially intact central filament 14 may be designed to approximate the mechanical properties of the arterial wall against which the stent material bears, so that the artery behaves in a normal, substantially anatomical manner indefinitely. Accordingly, no further surgical intervention would be required to remove the final stent structure comprised only of the central filament wires. As noted above, the material used for central filament 14 may also be designed to slowly degrade after being endothelialized, such that the filament framework left after t=3 is also eventually resorbed while minimizing embolic risk.

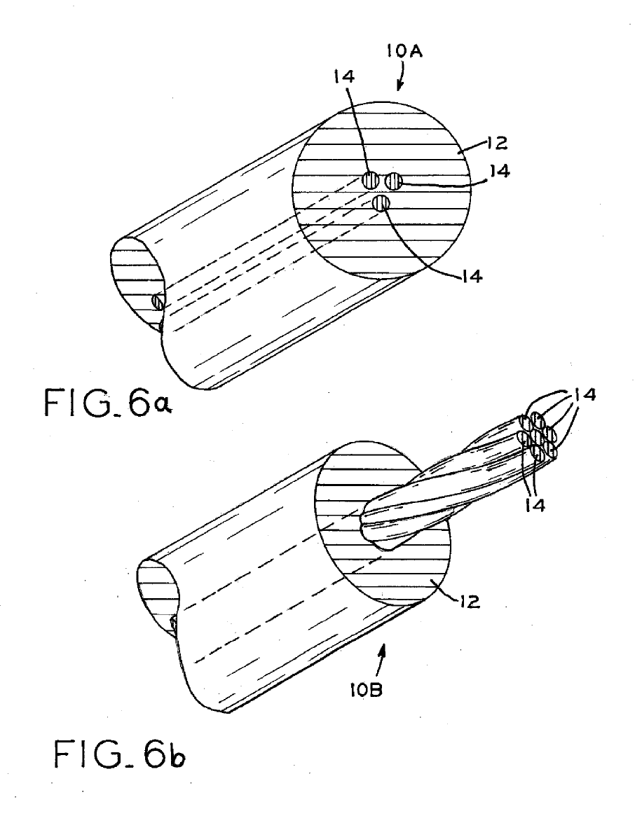

Turning now to FIGS. 6a and 6b, a plurality of central filaments 14 are shown surrounded by shell 12 to create alternative wire structures 10A and 10B. Wires 10A and 10B may be made by the same design principles and constraints and wire 10 described above, and descriptions of the structures and functions of wire 10 applies equally to wires 10A and 10B.

In FIG. 6a, multiple (as illustrated, three) filaments 14 are positioned in shell 12 to form wire 10A. In the illustrated embodiment, filaments 14 are all equally spaced from one another and are all equally spaced from the longitudinal axis of shell 12. Each filament 14 is straight, such that the longitudinal axes defined by filaments 14 are parallel to one another, and to the longitudinal axis of shell 12. To manufacture wire 10A, a precursor to filaments 14 may be placed into holes formed in a parent material, which in turn is a precursor to shell 12. The resulting assembly is then drawn down to overall diameter D2S in accordance with the description above. Further description of a manufacturing method that can be used to form wire 10A can be found in U.S. Pat. Nos. 7,020,947 and 7,490,396, both entitled METAL WIRE WITH FILAMENTS FOR BIOMEDICAL APPLICATIONS, the entire disclosures of which are hereby expressly incorporated by reference herein for all that they teach and for all purposes.

FIG. 6b illustrates wire 10B, in which multiple filaments 14 are formed into cable 16 disposed within shell 12. In the depicted embodiment, cable 16 is made from seven individual filaments 14 of a common size and constituency, wound into a spiral shape. In alternative embodiments, cable 16 may be made with more or fewer filaments 14, and filaments 14 may have common or varying sizes and constituencies.

For both wires 10A and 10B, the multiple filaments 14 used in shell 12 facilitate greater “purchase” of the parent material of shell 12 upon the matrix of filaments 14 matrix, thereby holding the any fragments of shell 12 in place on the filament during degradation (as shown in FIG. 3 and described above).

4. Applications—Stents

As described above, a primary application for wire 10 is stents, such as braided stent 100 shown in FIG. 8 and woven stent 110 shown in FIG. 9.

For exemplary stent applications, wire 10 is designed to provide a given initial flexural rigidity R₀ and to generally minimize flexural rigidity R₂ while maintaining a self-supporting structure of the matrix of filaments 14 which remain after shell 12 is fully biodegraded. For most stent applications, core ratio X is as little as 1%, 2% or 3% and as large as 8%, 9% or 10%, or may be any ratio within any range defined by any of the foregoing percentages, such as, for example, 1% to 10%, 2% to 9% or 3% to 8%.

For arterial stents and aortic devices designed for use in vena cava, wire 10 may have an overall diameter D_{2S} of up to 500 microns. For graft stents used in the abdominal area, wire 10 may have an overall diameter D_{2S} of between 100-500 microns. For superficial femoral stents used in branches of the main femoral artery, and infra-inguinal stents, wire 10 may have an overall diameter D_{2S} of between 100-400 microns. For iliac stenting applications, wire 10 may have an overall diameter D_{2S} of between 100-400 microns or, in some cases, up to 500 microns.

As described generally above, the overall geometry and material choices for wire 10 may reflect the intended use and desired degradation profile. For example, in the case stents 100 or 110 (FIGS. 8 and 9), it may be desirable to choose a size and material for shell 12 that can be expected to biodegrade over the course of at least three months for a patient that does not receive blood-thinning drugs. This, in turn, ensures that filament 14 will remain intact for at least the three-month period and prevent any dislodging of portions of shell 12. At the end of the three-month period, the patient's own vessel wall can be expected to endothelialize the material of wire 10, thereby naturally avoiding embolic risk as filament 14 begins to degrade. In other cases, where anti-platelet and/or anti cell-profilerative drugs are used after implantation of stent 100 or 110, the material and geometry of shell 12 may be selected to biodegrade over the course of at a year or more to allow for longer expected endothelialization of wire 10 by the adjacent cell wall.

5. Applications—Filters

Wire 10 may also be used for filters used, e.g., to arrest the downstream flow of solid materials in the bloodstream. For embolic filters used in the vena cava, wire 10 may have an overall diameter D_{2S} of between 100-400 microns. For coronary applications, wire 10 may have an overall diameter D_{2S} of between 75-200 microns. For neurovascular applications, wire 10 may have an overall diameter D_{2S} of between 15-100 microns.

Advantageously, filters made from wire 10 can mitigate embolic risk from foreign debris while avoiding any additional risk from debris formed from wire 10 itself. Similar benefits may be realized for aneurysm occlusion in the neurovascular area.

6. Applications—Tissue Joining

Wire 10 may also be used for sutures, staples and cables used, e.g., for joining and/or holding skin or tissue after an injury or surgery. In certain suture tissue joining applications, such as for the large incisions made in sternotomy procedures, the overall diameter of wire 10 may be as large as 1.5 mm.

While this invention has been described as having an exemplary design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A wire material comprising:
a filament made from a filament material;
a shell surrounding the filament and having a diameter less than 1.5 mm, the shell formed from a shell material, the shell material formed from a biodegradable material having a biodegradation rate faster than the filament material, the wire defining a non-biodegraded state including both the filament and the shell and a biodegraded state including only the substantially intact filament,
the wire defining a first flexural rigidity in the non-biodegraded state and a second flexural rigidity in the biodegraded state, the first flexural rigidity being at least two orders of magnitude larger than the second flexural rigidity, whereby the flexibility of the wire increases as the shell biodegrades.

2. The wire of claim 1, wherein the diameter of the shell is less than 500 μm.

3. The wire of claim 1, wherein the filament has a diameter less than one-half the diameter of the shell.

4. The wire of claim 1, wherein the first flexural rigidity is at least six orders of magnitude larger than the second flexural rigidity.

5. The wire of claim 1, wherein an expected total degradation time of the filament material is between 2% and 30% an expected total degradation time of the shell material, whereby the shell is adapted to substantially completely biodegrade before the filament experiences a significant loss of mass.

6. The wire of claim 5, wherein the respective materials and expected total degradation times of the shell and filament are configured such that the shell substantially completely biodegrades before the filament experiences 5% loss of mass.

7. The wire of claim 1, wherein the filament material of the filament is made from a non-biodegradable material.

8. The wire of claim 7, wherein the filament material is one of stainless steel, tantalum, nickel titanium, Co—Ni—Cr—Mo alloy, platinum, palladium, titanium, beta-titanium, alloys thereof, and high strength non-biodegradable polymer.

9. The wire of claim 1, wherein the filament material is made from a biodegradable material.

10. The wire of claim 9, wherein the filament material is one of Fe—Mn and an Mg alloy.

11. The wire of claim 1, wherein the shell material of the shell is one of ZM21, WE43, Mg and its alloys, Fe and its alloys, Fe—Mn and Zn and its alloys.

12. The wire of claim 1, wherein the filament is one of a plurality of filaments surrounded by the shell.

13. The wire of claim 12, wherein the plurality of filaments are spaced from one another and parallel to one another.

14. The wire of claim 12, wherein the plurality of filaments form a multi-filament twisted cable.

15. The wire of claim 1, wherein the filament is centrally located within the shell such that the longitudinal axes of the filament and the shell are coaxial.

16. A medical implant device including the wire of claim 1.

17. The medical implant device of claim 16, wherein the device comprises a stent.

18. The medical implant device of claim 16, wherein the device comprises a filter.

19. The medical implant device of claim 16, wherein the device comprises a suture.

20. The wire of claim 1, wherein the shell comprises a substantially cylindrical outer surface having at least one non-cylindrical irregularity formed therein, such that the irregularity is susceptible to crevice-type corrosion to promote biodegradation in and around the irregularity.

21. The wire of claim 1, wherein the shell comprises at least one coated portion having an anti-degradation coating applied thereto, such that the coated portion is adapted to experience slower biodegradation as compared to uncoated portions.

22. The wire of claim 21, wherein the coating comprises at least one of an oxide, a polymer and a ceramic.

23. The wire of claim 21, wherein the coating comprises one of PGA and PLLA.

24. The wire of claim 1, wherein the filament comprises a shape-set NiTi having a first configuration and the shell is formed in a second configuration different from the first configuration, whereby the shape-set NiTi reconfigures from the second configuration in the non-biodegraded state to the first configuration in the biodegraded state.
